(19) United States
(12) Patent Application Publication
(10) Pub. No.: US 2025/0064776 A1
(43) Pub. Date: Feb. 27, 2025

(54) TREATMENT OF AMYOTROPHIC LATERAL SCLEROSIS USING PKC ACTIVATORS

(71) Applicant: Synaptogenix, Inc., New York, NY (US)

(72) Inventor: Daniel L. ALKON, Chevy Chase, MD (US)

(73) Assignee: Synaptogenix, Inc., New York, NY (US)

(21) Appl. No.: 18/944,619

(22) Filed: Nov. 12, 2024

Related U.S. Application Data
(62) Division of application No. 17/551,493, filed on Dec. 15, 2021.
(60) Provisional application No. 63/126,020, filed on Dec. 16, 2020.

(51) Int. Cl. A61K 31/365; A61K 9/00; A61K 31/215; A61K 38/18; A61K 38/30; A61P 25/28

(52) U.S. Cl. CPC A61K 31/365; A61K 9/0019; A61K 31/215; A61K 38/1833; A61K 38/185; A61K 38/30; A61P 25/28

(57) ABSTRACT

A method for treating or preventing amyotrophic lateral sclerosis (ALS) or other motor neuron disease in a subject, the method comprising administering to the subject a PKC activating compound (e.g., a bryostatin, such as bryostatin-1, or a bryolog) in a therapeutically effective amount to treat or prevent the motor neuron disease by activating PKC in the subject. The ALS may be, for example, classical ALS, primary lateral sclerosis (PLS), progressive muscular atrophy (PMA), and progressive bulbar palsy (PBP). The PKC activating compound may be administered at an initial loading dose of about 15, 24, or 48 micrograms weekly in the first one week or consecutive two or three weeks, followed by doses of about 12, 20, or 40 micrograms alternately every two or three weeks for at least 4, 5, 6, 8, 10, 12, 14, 16, 18, 20, 24, or 30 total weeks.

### CROSS REFERENCE TO RELATED APPLICATION

The present application claims benefit of U.S. Provisional Application No. 63/126,020, filed on Dec. 16, 2020, all of the contents of which are incorporated herein by reference.

### BACKGROUND

Amyotrophic lateral sclerosis (ALS), also known as Lou Gehrig's Disease, is a relatively rare yet highly debilitating neurological syndrome characterized by progressive degeneration of motor neurons of the spinal cord, medulla, and cortex. As the disease targets motor neurons, ALS is marked by progressive muscular weakness eventually leading to atrophy and loss in voluntary muscle movement. Spasticity and hyperreflexia generally also accompany the muscular deterioration. In the late stages of the disease, those afflicted with ALS experience loss in ability to speak, eat, walk, and eventually, even breathe. In some cases, cells of the motor cranial nuclei in the medulla are targeted by the disease, in which case the disease may be referred to as progressive bulbar palsy. The onset is fairly rapid and the prognosis is generally very poor. There are, at present, no known effective medical interventions. Thus, a method for mitigating the progression of ALS would be a significant and desperately needed advance in the treatment of this cruel disease.

### SUMMARY

The present disclosure is directed to a method for treating or preventing amyotrophic lateral sclerosis (ALS) or other motor neuron disease by administering a PKC activating compound in a therapeutically effective amount to a subject in need thereof to result in treatment (e.g., mitigation) or prevention of symptoms of the motor neuron disease. The ALS may be, more particularly, classical ALS, primary lateral sclerosis (PLS), progressive muscular atrophy (PMA), or progressive bulbar palsy (PBP). The PKC activating compound may be, for example, a macrocyclic lactone compound, such as a bryostatin compound (e.g., bryostatin-1) or bryolog compound, a polyunsaturated fatty acid (PUFA) or cyclopropanated or epoxidized derivative thereof, or a growth factor (e.g., BDNF, HGF, NGF, and IGF).

In some embodiments, the PKC activating compound is administered in an initial loading dose that is 15-25% greater in dosage than successive weekly dosages. In a first embodiment, the PKC activating compound is administered at an initial loading dose of about 15 micrograms per week for two consecutive weeks followed by about 12 micrograms on alternate weeks for a least four, six, eight, ten, or twelve weeks. In a second embodiment, the PKC activating compound is administered at an initial loading dose of about 24 micrograms per week for two consecutive weeks followed by about 20 micrograms on alternate weeks for at least four weeks. In a third embodiment, the PKC activating compound is administered at an initial loading dose of about 48 micrograms per week for two consecutive weeks followed by about 40 micrograms on alternate weeks for a least four, six, eight, ten, or twelve weeks.

### DETAILED DESCRIPTION

As used herein, the singular forms “a,” “an,” and “the” include plural reference.

As used herein, “protein kinase C activator” or “PKC activator” refers to a substance that increases the rate of the reaction catalyzed by PKC. PKC activators can be non-specific or specific activators. A specific activator activates one PKC isoform, e.g., PKC-ε (epsilon), to a greater detectable extent than another PKC isoform. Protein kinase C (PKC) is one of the largest gene families of protein kinase. Several PKC isozymes are expressed in the brain, including PKCα, PKCβ1, PKCβII, PKCδ, PKCε, and PKCγ. PKC is primarily a cytosolic protein, but with stimulation it translocates to the membrane.

PKC activators have been associated with prevention and treatment of various diseases and conditions. For example, PKC has been shown to be involved in numerous biochemical processes relevant to AD, and PKC activators have demonstrated neuroprotective activity in animal models of AD. PKC activation has a crucial role in learning and memory enhancement, and PKC activators have been shown to increase memory and learning (Sun and Alkon, Eur J Pharmacol. 2005;512:43-51; Alkon et al., Proc Natl Acad Sci USA. 2005;102:16432-16437). PKC activation also has been shown to induce synaptogenesis in rat hippocampus, suggesting the potential of PKC-mediated antiapoptosis and synaptogenesis during conditions of neurodegeneration (Sun and Alkon, Proc Natl Acad Sci USA. 2008; 105(36): 13620-13625). In fact, synaptic loss appears to be a pathological finding in the brain that is closely correlated with the degree of dementia in AD patients. PKC activation has further been shown to protect against traumatic brain injury-induced learning and memory deficits, (Zohar et al., Neurobiology of Disease, 2011, 41:329-337), has demonstrated neuroprotective activity in animal models of stroke, (Sun et al., Eur. J. Pharmacol., 2005, 512:43-51), and has shown neuroprotective activity in animal models of depression, (Sun et al., Eur. J. Pharmacol., 2005, 512:43-51).

Neurotrophins, particularly brain-derived neurotrophic factor (BDNF) and nerve growth factor (NGF), are key growth factors that initiate repair and regrowth of damaged neurons and synapses. Activation of some PKC isoforms, particularly PKCε and PKCα, protect against neurological injury, most likely by upregulating the production of neurotrophins such as BDNF (Weinreb et al., FASEB Journal. 2004; 18:1471-1473). The activation of PKCε also increases brain postsynaptic density anchoring protein (PSD-95) which is an important marker for synaptogenesis.

In addition, changes in dendritic spine density form the basis of learning- and memory-induced changes in synaptic structure that increase synaptic strength. Abnormalities in the number and morphology of dendritic spines have been observed in many cognitive disorders, such as attention deficit hyperactivity disorder, schizophrenia, autism, mental retardation, and fragile X syndrome. For example, the brains of schizophrenic patients and people suffering from cognitive-mood disorders show a reduced number of dendritic spines in the brain areas associated with these diseases. In mental retardation and autism, the shapes of the dendritic spines are longer and appear more immature.

As used herein, the term “fatty acid” refers to a compound composed of a hydrocarbon chain and ending in a free acid, an acid salt, or an ester. When not specified, the term “fatty acid” is meant to encompass all three forms. Those skilled in the art understand that certain expressions are interchangeable. For example, “methyl ester of linolenic acid” is the same as “linolenic acid methyl ester,” which is the same as “linolenic acid in the methyl ester form.”

As used herein, the term “cyclopropanated” or “CP” refers to a compound wherein at least one carbon-carbon double bond in the molecule has been replaced with a cyclopropane group. The cyclopropyl group may be in cis or trans configuration. Unless otherwise indicated, the cyclopropyl group is in the cis configuration. Compounds with multiple carbon-carbon double bonds have many cyclopropanated forms. For example, a polyunsaturated compound in which only one double bond has been cyclopropanated is herein referred to as being in “CP1 form.” Similarly, “CP6 form” indicates that six double bonds are cyclopropanated. Docosahexaenoic acid (“DHA”) methyl ester has six carbon-carbon double bonds, and thus, can have 1-6 cyclopropane rings.

Shown below are the CP1 and CP6 forms. With respect to compounds that are not completely cyclopropanated (e.g. DHA-CP1), the cyclopropane group(s) can occur at any of the carbon-carbon double bonds.

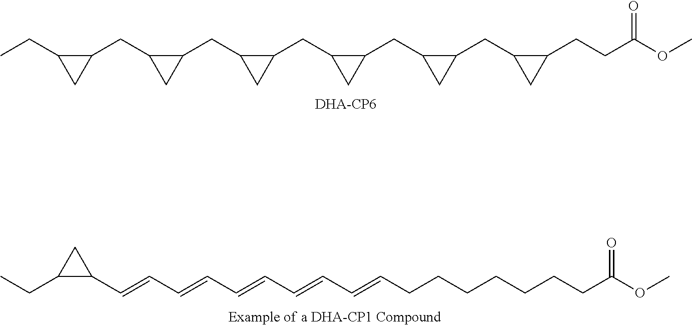

As used herein, the term “HGF activator” refers to a substance that increases the rate of the reaction catalyzed by HGF. HGF is well known in the art, as described in, for example, T. Nakamura et al., Proc., Jpn. Acad. Ser. B Phys. Biol. Sci., 86(6), 588-610, 2010.

As used herein, the term “cholesterol” refers to cholesterol and derivatives thereof. For example, “cholesterol” may or may not include the dihydrocholesterol species.

As used herein, the word “synaptogenesis” refers to a process involving the formation of synapses.

As used herein, the word “synaptic networks” refer to a multiplicity of neurons and synaptic connections between the individual neurons. Synaptic networks may include extensive branching with multiple interactions. Synaptic networks can be recognized, for example, by confocal visualization, electron microscopic visualization, and electrophysiologic recordings.

The phrase “pharmaceutically acceptable” refers to molecular entities and compositions that are physiologically tolerable and do not typically produce adverse reactions when administered to a subject. The pharmaceutically acceptable substance is typically approved by a regulatory agency or listed in the U.S. Pharmacopeia or other generally recognized pharmacopeia for use in animals, and more particularly in humans. The term “pharmaceutically acceptable carrier” generally refers to a chemical substance in which the active ingredient may be combined and which, following the combination, can be used to administer the active ingredient to a subject. The carrier can also be, for example, a diluent, adjuvant, excipient, or vehicle for the compound being administered.

The term “therapeutically effective amount” refers to an amount of a therapeutic agent that results in a measurable or observable therapeutic response. A therapeutic response may be, for example, any response that a person of sound medical adjustment (e.g., a clinician or physician) will recognize as an effective response to the therapy, including improvement of symptoms and surrogate clinical markers. Thus, a therapeutic response will generally be a mitigation, amelioration, or inhibition of one or more symptoms of the motor neuron disease. A measurable therapeutic response also includes a finding that a symptom or disease is prevented or has a delayed onset, or is otherwise attenuated by the therapeutic agent.

The term “subject,” as used herein, refers to a human or other mammal in need of treatment with the PKC activating compound. The subject may be, for example, a human having a motor neuron disease, particularly ALS. Some examples of mammals other than humans that may be treated include dogs, cats, monkeys, and apes.

The terms “approximately” and “about” mean to be nearly the same as a referenced number or value including an acceptable degree of error for the quantity measured given the nature or precision of the measurements. As used herein, the terms “approximately” and “about” are generally understood to encompass ±20% or ±10% of a specified amount, frequency or value. Numerical quantities given herein are approximate unless stated otherwise, meaning that the term “about” or “approximately” can be inferred when not expressly stated. For example, the term “about 20 μg” may be interpreted as precisely that amount or as being within a margin of 16-24 μg or 18-22 μg.

The terms “administer,” “administration,” or “administering,” as used herein, refer to (1) providing, giving, dosing and/or prescribing by either a health practitioner or his/her authorized agent or under his/her direction a composition according to the disclosure, and (2) putting into, taking, or consuming by the patient or person himself or herself, a composition according to the disclosure. As used herein, “administration” of a composition includes any route of administration, including oral, intravenous, subcutaneous, intraperitoneal, and intramuscular.

The phrase “weekly dosing regimen” is used when the subject is administered a dose of a therapeutic agent (drug) every week for a predetermined number of consecutive weeks. For example, the subject may receive a single dose of a therapeutic agent each week for three consecutive weeks.

The phrases “spaced dosing regimen” and “intermittent dosing regimen” are herein used interchangeably and refer to an on/off dosing regimen of a defined periodicity. In some embodiments, a spaced dosing regimen or intermittent dosing regimen may be used for administering a PKC activating compound to a subject. The spaced or intermittent dosing regimen may entail, for example, administering a PKC activating compound to the subject once a week for two or three consecutive weeks, followed by cessation of administration or dosing for two or three consecutive weeks. In further embodiments, the administration may continue in alternating intervals of administering the PKC activator once a week for two or three consecutive weeks, followed by cessation of administration or dosing for two or three consecutive weeks, and continuing those alternating intervals over a period of about 4 months, about 8 months, about 1 year, about 2 years, about 5 years, or otherwise for the duration of therapy with the PKC activator.

The PKC activator may be administered according to any suitable dosing schedule or regimen. In some embodiments, the PKC activator, such as a bryostatin (e.g., bryostatin-1), may be administered in an amount ranging from about 0.01 μg/m² to about 100 μg/m². In different embodiments, the amount administered is precisely, about, up to, or less than 0.01 μg/m², 0.05 μg/m², 0.1 μg/m², 0.5 μg/m², 1 μg/m², 5 μg/m², 10 μg/m², 15 μg/m², 20 μg/m², 25 μg/m², 30 μg/m², 35 μg/m², 40 μg/m², 45 μg/m², 50 μg/m², 55 μg/m², 60 μg/m², 65 μg/m², 70 μg/m², 75 μg/m², 80 μg/m², 85 μg/m², 90 μg/m², 95 μg/m², or 100 μg/m², or an amount within a range bounded by any two of the foregoing amounts, e.g., 0.01-100 μg/m², 0.1-100 μg/m², 1-100 μg/m², 5-100 μg/m², 10-100 μg/m², 0.01-50 μg/m², 0.1-50 μg/m², 1-50 μg/m², 5-50 μg/m², 10-50 μg/m², 0.01-20 μg/m², 0.1-20 μg/m², 1-20 μg/m², 5-20 μg/m², or 10-20 μg/m². In particular embodiments, the amount may range from about 10-40 μg/m², or more particularly, about 15 μg/m², about 20 μg/m², about 25 μg/m², about 30 μg/m², about 35 μg/m², or about 40 μg/m², or about 45 μg/m², or about 50 μg/m², or an amount within a range bounded by any two of the foregoing values. Notably, any of the amounts above or below expressed as “μg/m²” may alternatively be interpreted in terms of micrograms (μg) or micrograms per 50 kg body weight “μg/50 kg”. For example, 25 μg/m² may be interpreted as 25 μg or 25 μg/50 kg.

In some embodiments, the PKC activator is administered as a dose in the range of about 0.01 to 100 μg/m²/week. For example, the dose may be administered each week in a range of about 0.01 to about 25 μg/m²/week; about 1 to about 20 μg/m²/week, about 5 to about 20 μg/m²/week, or about 10 to about 20 μg/m²/week. In particular embodiments, the dose may be about or less than, for example, 5 μg/m²/week, 10 μg/m²/week, 15 μg/m²/week, 20 μg/m²/week, 25 μg/m²/week, or 20 μg/m²/week. Any of the foregoing dosages may be administered over a suitable time period, e.g., three weeks, four weeks, (approximately 1 month), two months, three months (approximately 12 or 13 weeks), four months, five months, six months, or a year. Notably, any of the amounts above or below expressed as “μg/m²” may alternatively be interpreted in terms of micrograms (μg) or micrograms per 50 kg body weight “μg/50 kg”.

In some embodiments, the PKC activator (e.g., a bryostatin) is administered in an amount of precisely or about 20 μg, 30 μg, or 40 μg (20 μg/m², 30 μg/m², or 40 μg/m²) every week or every two weeks for a total period of time of, e.g., four weeks, (approximately 1 month), five weeks, six weeks, eight weeks, ten weeks, twelve weeks, four months, five months, six months, or a year. The administration may alternatively start with an initial single higher amount (e.g., 10%, 15%, 20%, or 25% higher amount than successive administrations). For example, in some embodiments, the PKC activator may be administered in an amount of precisely or about 15 μg, 24 μg, or 48 μg for the first week or first two or three consecutive weeks followed by administrations of 12 μg, 20 μg or 40 μg, respectively, every week or alternately every two or three weeks for at least four weeks (approximately 1 month), six weeks, eight weeks, ten weeks, twelve weeks, fifteen weeks, eighteen weeks, or for at least three months, four months, five months, six months, or a year. The term “alternately,” as used herein, indicates a period of time in which the PKC activator is not being administered. For example, “alternately every two or three weeks” indicates, respectively, regular one-week periods of no administration or regular two-week periods of no administration (also referred to herein as “1 on/1 off” and “1 on/2 off” dosing regimens. Other alternating dosing regimens are possible, including, for example, “2 on/1 off”, “2 on/2 off”, “1 on/3 off”, “2 on/3 off”, “3 on/3 off”, “3 on/1 off”, and “3 on/2 off”. Notably, any of the amounts above or below expressed as μg may alternatively be interpreted in terms of μg/m2 or micrograms per 50 kg body weight “μg/50 kg”.

In some embodiments, the PKC activator is selected from macrocyclic lactones, bryologs, diacylglycerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, and fatty acid esters.

In particular embodiments, the PKC activator is a macrocyclic lactone selected from bryostatin and neristatin, such as neristatin-1. In further embodiments, the PKC activator is bryostatin, such as bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18. In a further embodiment, the PKC activator is bryostatin-1.

In a further aspect, the role of such intermittent dosing of a PKC activator on restoring or upregulating BDNF, increasing the postsynaptic density of the anchoring protein PSD-95, and lowering or preventing the downregulation of PCK-ε, is disclosed.

In some embodiments, the therapeutically effective amount of PKC activator is administered according to any suitable dosing schedule or regimen described. In some embodiments, administration of the PKC activator results in an increase in the number of fully mature mushroom spine synapses. In other embodiments, administration of the PKC activator results in at least partial or full restoration of mature mushroom spines or mushroom spine synapses.

In different embodiments, the PKC activator is selected from macrocyclic lactones, bryologs, diacylglycerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acid alcohols and derivatives, and fatty acid esters. In particular embodiments, the PKC activator is a macrocyclic lactone selected from bryostatins and neristatin, such as neristatin-1. In a further embodiment, the PKC activator is a bryostatin, such as bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18. In a further embodiment, the PKC activator is bryostatin-1. In one embodiment, the therapeutically effective amount of the PKC activator, such as bryostatin-1, is about 25 μg/m².

In some embodiments, the PKC activator is a macrocyclic lactone. Macrocyclic lactones (also known as macrolides) generally comprise 14-, 15-, or 16-membered lactone rings. Macrolides belong to the polyketide class of natural products. Macrocyclic lactones and derivatives thereof are described, for example, in U.S. Pat. Nos. 6,187,568; 6,043,270; 5,393,897; 5,072,004; 5,196,447; 4,833,257; and 4,611,066; and 4,560,774; each incorporated by reference herein in its entirety. Those patents describe various compounds and various uses for macrocyclic lactones including their use as an anti-inflammatory or anti-tumor agents. See also Szallasi et al. J. Biol. Chem. (1994), vol. 269, pp. 2118-2124; Zhang et al., Cancer Res. (1996), vol. 56, pp. 802-808; Hennings et al. Carcinogenesis (1987), vol. 8, pp. 1343-1346; Varterasian et al. Clin. Cancer Res. (2000), vol. 6, pp. 825-828; Mutter et al. Bioorganic & Med. Chem. (2000), vol. 8, pp. 1841-1860; each incorporated by reference herein in its entirety. In particular embodiments, the macrocyclic lactone is a bryostatin. Bryostatins include, for example, Bryostatin-1, Bryostatin-2, Bryostatin-3, Bryostatin-4, Bryostatin-5, Bryostatin-6, Bryostatin-7, Bryostatin-8, Bryostatin-9, Bryostatin-10, Bryostatin-11, Bryostatin-12, Bryostatin-13, Bryostatin-14, Bryostatin-15, Bryostatin-16, Bryostatin-17, and Bryostatin-18.

In one embodiment. the bryostatin is Bryostatin-1 (shown below).

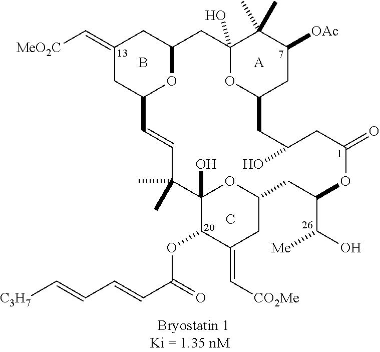

In another embodiment, the bryostatin is Bryostatin-2 (shown below; R=COC₇Hn₁₁, R′=H).

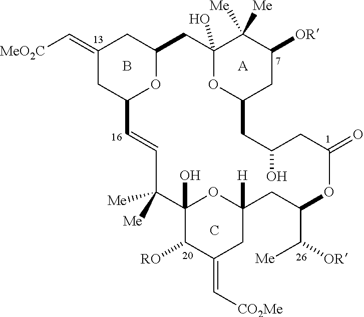

In another embodiment, the macrocyclic lactone is a neristatin, such as neristatin-1. In another embodiment, the macrocyclic lactone is selected from macrocyclic derivatives of cyclopropanated PUFAs such as, 24-octaheptacyclononacosan-25-one (cyclic DHA-CP6) (shown below).

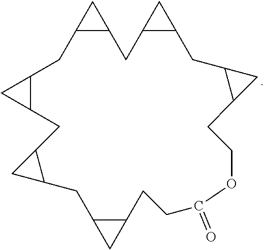 [SMILES: O=C1CCC2CC2CC2CC2CC2CC2CC2CC2CC2CC2CC2CC2CCO1]

In another embodiment, the macrocyclic lactone is a bryolog, wherein bryologs are analogues of bryostatin. Bryologs can be chemically synthesized or produced by certain bacteria. Different bryologs exist that modify, for example, the rings A, B, and C (see Bryostatin-1, figure shown above) as well as the various substituents. As a general overview, bryologs are considered less specific and less potent than bryostatin but are easier to prepare.

Table 1 summarizes structural characteristics of several bryologs and their affinity for PKC (ranging from 0.25 nM to 10 μM). While Bryostatin-1 has two pyran rings and one 6-membered cyclic acetal, in most bryologs one of the pyrans of Bryostatin-1 is replaced with a second 6-membered acetal ring. This modification may reduce the stability of bryologs, relative to Bryostatin-1, for example, in strong acid or base, but has little significance at physiological pH. Bryologs also tend to have a lower molecular weight (ranging from about 600 g/mol to 755 g/mol), as compared to Bryostatin-1 (988), a property which may facilitate transport across the blood-brain barrier.

TABLE 1
Bryologs

| | PKC Affin | | |
|---|---|---|---|
| Name | (nM) | MW | Description |
| Bryostatin-1 | 1.35 | 988 | 2 pyran + 1 cyclic acetal + macrocycle |
| Analog 1 | 0.25 | 737 | 1 pyran + 2 cyclic acetal + macrocycle |
| Analog 2 | 6.50 | 723 | 1 pyran + 2 cyclic acetal + macrocycle |
| Analog 7a | — | 642 | 1 pyran + 2 cyclic acetals + macrocycle |
| Analog 7b | 297 | 711 | 1 pyran + 2 cyclic acetals + macrocycle |
| Analog 7c | 3.4 | 726 | 1 pyran + 2 cyclic acetals + macrocycle |
| Analog 7d | 10000 | 745 | 1 pyran + 2 cyclic acetals + macrocycle, |
| | | | acetylated |
| Analog 8 | 8.3 | 754 | 2 cyclic acetals + macrocycle |
| Analog 9 | 10000 | 599 | 2 cyclic acetals |

Analog 1 exhibits the highest affinity for PKC. Wender et al., Curr. Drug Discov. Technol. (2004), vol. 1, pp. 1-11; Wender et al. Proc. Natl. Acad. Sci. (1998), vol. 95, pp. 6624-6629; Wender et al., J. Am. Chem. Soc. (2002), vol. 124, pp. 13648-13649, each incorporated by reference herein in their entireties. Only Analog 1 exhibits a higher affinity for PKC than

Bryostatin-1. Analog 2. which lacks the A ring of Bryostatin-1. is the simplest analog that maintains high affinity for PKC. In addition to the active bryologs. Analog 7d. which is acetylated at position 26. has virtually no affinity for PKC.

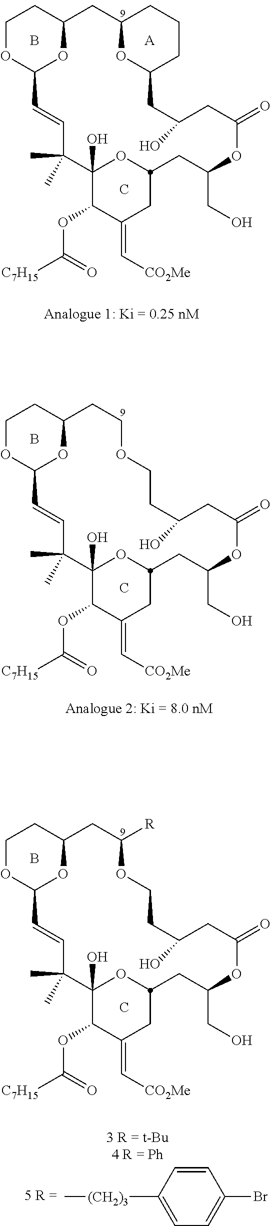

B-ring bryologs may also be used in the present disclosure. These synthetic bryologs have affinities in the low nanomolar range. Wender et al., Org Lett. (2006), vol. 8, pp. 5299-5302, incorporated by reference herein in its entirety. B-ring bryologs have the advantage of being completely synthetic, and do not require purification from a natural source.

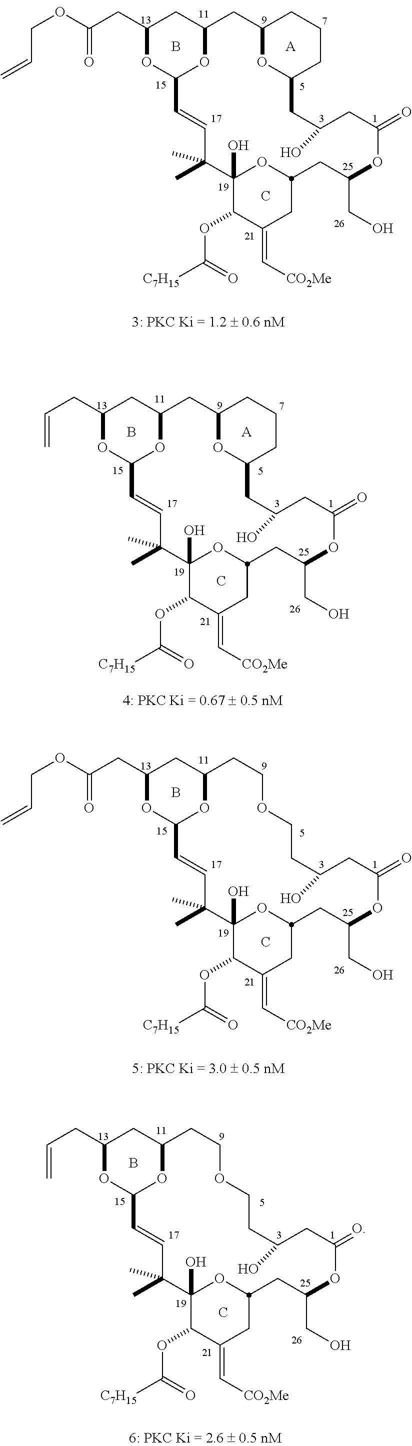

A third class of suitable bryostatin analogs are the A-ring bryologs. These bryologs have slightly lower affinity for PKC than Bryostatin-1 (6.5 nM, 2.3 nM, and 1.9 nM for bryologs 3, 4, and 5, respectively) and a lower molecular weight. A-ring substituents are important for non-tumorigenesis.

Bryostatin analogs are described, for example, in U.S. Pat. Nos. 6,624,189 and 7,256,286. Methods using macrocyclic lactones to improve cognitive ability are also described in U.S. Pat. No. 6,825,229 B2.

The PKC activator may also include derivatives of diacylglycerols (DAGs). See, e.g., Niedel et al., Proc. Natl. Acad. Sci. (1983), vol. 80, pp. 36-40; Mori et al., J. Biochem. (1982), vol. 91, pp. 427-431; Kaibuchi et al., J. Biol. Chem. (1983), vol. 258, pp. 6701-6704. Activation of PKC by diacylglycerols is transient, because they are rapidly metabolized by diacylglycerol kinase and lipase. Bishop et al. J. Biol. Chem. (1986), vol. 261, pp. 6993-7000; Chuang et al. Am. J. Physiol. (1993), vol. 265, pp. C927-C933; incorporated by reference herein in their entireties. The fatty acid substitution on the diacylglycerol derivatives may determine the strength of activation. Diacylglycerols having an unsaturated fatty acid may be most active. The stereoisomeric configuration is important; fatty acids with a 1,2-sn configuration may be active while 2,3-sn-diacylglycerols and 1,3-diacylglycerols may not bind to PKC. Cis-unsaturated fatty acids may be synergistic with diacylglycerols. In some embodiments, the PKC activator excludes DAG or DAG derivatives.

The PKC activator may also include isoprenoids. Farnesyl thiotriazole, for example, is a synthetic isoprenoid that activates PKC with a K_{d} of 2.5 μM. Farnesyl thiotriazole, for example, is equipotent with dioleoylglycerol, but does not possess hydrolyzable esters of fatty acids. Gilbert et al., Biochemistry (1995), vol. 34, pp. 3916-3920; incorporated by reference herein in its entirety. Farnesyl thiotriazole and related compounds represent a stable, persistent PKC activator. Because of its low molecular weight (305.5 g/mol) and absence of charged groups, farnesyl thiotriazole may readily cross the blood-brain barrier.

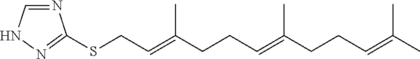 [SMILES: CC(C)=CCC/C(C)=C/CC/C(C)=C/CSc1nc[nH]n1]

Yet other types of PKC activators include octylindolactam V, gnidimacrin, and ingenol. Octylindolactam V is a non-phorbol protein kinase C activator related to teleocidin. The advantages of octylindolactam V (specifically the (-)-enantiomer) may include greater metabolic stability, high potency (EC₅₀=29 nM) and low molecular weight that facilitates transport across the blood brain barrier. Fujiki et al. Adv. Cancer Res. (1987), vol. 49 pp. 223-264; Collins et al. Biochem. Biophys. Res. Commun. (1982), vol. 104, pp. 1159-4166, each incorporated by reference herein in its entirety.

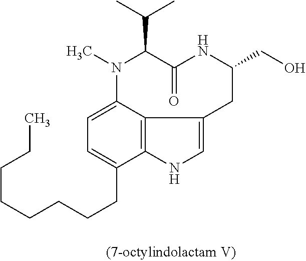 [SMILES: CCCCCCCCc1ccc2c3c(c[nH]c13)C[C@@H](CO)NC(=O)[C@H](C(C)C)N2C]

Gnidimacrin is a daphnane-type diterpene that displays potent antitumor activity at concentrations of 0.1 nM-1 nM against murine leukemias and solid tumors. It may act as a PKC activator at a concentration of 0.3 nM in K562 cells, and regulate cell cycle progression at the G1/S phase through the suppression of Cdc25A and subsequent inhibition of cyclin-dependent kinase 2 (Cdk2) (100% inhibition achieved at 5 ng/ml). Gnidimacrin is a heterocyclic natural product similar to Bryostatin-1, but somewhat smaller (MW=774.9 g/mol).

Iripallidal is a bicyclic triterpenoid isolated from Iris pallida. Iripallidal displays anti-proliferative activity in a NCI 60 cell line screen with GI₅₀ (concentration required to inhibit growth by 50%) values from micromolar to nanomolar range. It binds to PKCα with high affinity (Kᵢ=75.6 nM). It may induce phosphorylation of Erk1/2 in a RasGRP3-dependent manner. Its molecular weight is 486.7 g/mol. Iripallidal is about half the size of Bryostatin-1 and lacks charged groups.

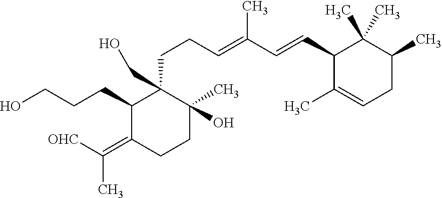

Ingenol is a diterpenoid related to phorbol but less toxic. It is derived from the milkweed plant Euphorbia peplus. Ingenol 3,20-dibenzoate, for example, competes with [3H] phorbol dibutyrate for binding to PKC (Kᵢ=240 nM). Winkler et al., J. Org. Chem. (1995), vol. 60, pp. 1381-1390, incorporated by reference herein. Ingenol-3-angelate exhibits antitumor activity against squamous cell carcinoma and melanoma when used topically. Ogbourne et al. Anticancer Drugs (2007), vol. 18, pp. 357-362, incorporated by reference herein.

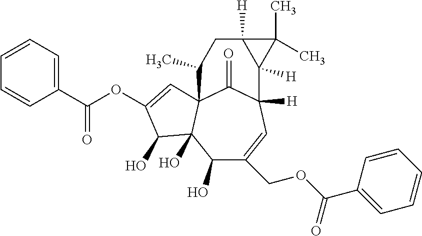 [SMILES: C[C@@H]1C[C@@H]2[C@H]([C@@H]3C=C(COC(=O)c4ccccc4)[C@@H](O)[C@]4(O)[C@@H](O)C(OC(=O)c5ccccc5)=C[C@@]14C3=O)C2(C)C]

The PKC activator may also include the class of napthalenesulfonamides, including N-(n-heptyl)-5-chloro-1-naphthalenesulfonamide (SC-10) and N-(6-phenylhexyl)-5-chloro-1-naphthalenesulfonamide. SC-10 may activate PKC in a calcium-dependent manner, using a mechanism similar to that of phosphatidylserine. Ito et al., Biochemistry (1986), vol. 25, pp. 4179-4184, incorporated by reference herein. Naphthalenesulfonamides act by a different mechanism than bryostatin and may show a synergistic effect with bryostatin or member of another class of PKC activators. Structurally, naphthalenesulfonamides are similar to the calmodulin (CaM) antagonist W-7, but are reported to have no effect on CaM kinase.

The PKC activator may also include the class of diacylglycerol kinase inhibitors, which indirectly activate PKC. Examples of diacylglycerol kinase inhibitors include, but are not limited to, 6-(2-(4-[(4-fluorophenyl)phenylmethylene]-1-piperidinyl) ethyl)-7-methyl-5H-thiazolo[3,2-a]pyrimidin-5-one (R59022) and [3-[2-[4-(bis-(4-fluorophenyl)methylene]piperidin-1-yl)ethyl]-2,3-dihydro-2-thioxo-4(1H)-quinazolinone (R59949).

The PKC activator may also be a growth factor, such as fibroblast growth factor 18 (FGF-18) and insulin growth factor, which function through the PKC pathway. FGF-18 expression is up-regulated in learning, and receptors for insulin growth factor have been implicated in learning. Activation of the PKC signaling pathway by these or other growth factors offers an additional potential means of activating PKC.

The PKC activator may also include hormones and growth factor activators, including 4-methyl catechol derivatives, such as 4-methylcatechol acetic acid (MCBA), which stimulate the synthesis and/or activation of growth factors, such as NGF and BDNF, which also activate PKC as well as convergent pathways responsible for synaptogenesis and/or neuritic branching.

The PKC activator may also include polyunsaturated fatty acids (“PUFAs”). These compounds are essential components of the nervous system and have numerous health benefits. In general, PUFAs increase membrane fluidity, rapidly oxidize to highly bioactive products, produce a variety of inflammatory and hormonal effects, and are rapidly degraded and metabolized. The inflammatory effects and rapid metabolism is likely the result of their active carbon-carbon double bonds.

In one embodiment, the PUFA is selected from linoleic acid (shown below).

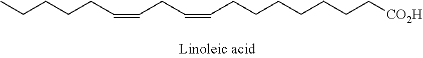 [SMILES: CCCCC/C=C\C/C=C\CCCCCCCC(=O)O]

The PKC activator may also be a PUFA or MUFA derivative. In particular embodiments, the PUFA or MUFA derivative is a cyclopropanated derivative. Certain cyclopropanated PUFAs, such as DCPLA (i.e., linoleic acid with cyclopropane at both double bonds), may be able to selectively activate PKC-ε. See Journal of Biological Chemistry, 2009, 284 (50): 34514-34521; see also U.S. Patent Application Publication No. 2010/0022645 A1. Like their parent molecules, PUFA derivatives are thought to activate PKC by binding to the PS site.

Cyclopropanated fatty acids exhibit low toxicity and are readily imported into the brain where they exhibit a long half-life (t_{1/2}). Conversion of the double bonds into cyclopropane rings prevents oxidation and metabolism to inflammatory byproducts and creates a more rigid U-shaped 3D structure that may result in greater PKC activation. Moreover, this U-shape may result in greater isoform specificity. For example, cyclopropanated fatty acids may exhibit potent and selective activation of PKC-ε.

The Simmons-Smith cyclopropanation reaction is an efficient way of converting double bonds to cyclopropane groups. This reaction, acting through a carbenoid intermediate, preserves the cis-stereochemistry of the parent molecule. Thus, the PKC-activating properties are increased while metabolism into other molecules, such as bioreactive eicosanoids, thromboxanes, or prostaglandins, is prevented.

A particular class of PKC-activating fatty acids is Omega-3 PUFA derivatives. In at least one embodiment, the Omega-3 PUFA derivatives are selected from cyclopropanated docosahexaenoic acid, cyclopropanated eicosapentaenoic acid, cyclopropanated rumelenic acid, cyclopropanated parinaric acid, and cyclopropanated linolenic acid (CP3 form shown below).

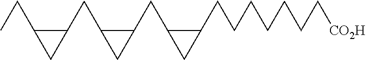 [SMILES: CCC1CC1CC1CC1CC1CC1CCCCCCCC(=O)O]

Another class of PKC-activating fatty acids is Omega-6 PUFA derivatives. In at least one embodiment, the Omega-6 PUFA derivatives are selected from cyclopropanated linoleic acid (“DCPLA,” CP2 form shown below),

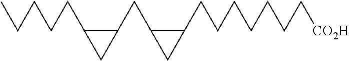 [SMILES: CCCCCC1CC1CC1CC1CCCCCCCC(=O)O]

cyclopropanated arachidonic acid, cyclopropanated eicosadienoic acid, cyclopropanated dihomo-gamma-linolenic acid, cyclopropanated docosadienoic acid, cyclopropanated adrenic acid, cyclopropanated calendic acid, cyclopropanated docosapentaenoic acid, cyclopropanated jacaric acid, cyclopropanated pinolenic acid, cyclopropanated podocarpic acid, cyclopropanated tetracosatetraenoic acid, and cyclopropanated tetracosapentaenoic acid.

Vernolic acid is a naturally occurring compound. However, it is an epoxyl derivative of linoleic acid and therefore, as used herein, is considered an Omega-6 PUFA derivative. In addition to vernolic acid, cyclopropanated vernolic acid (shown below) is an Omega-6 PUFA derivative.

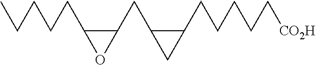 [SMILES: CCCCCC1OC1CC1CC1CCCCCC(=O)O]

Another class of PKC-activating fatty acids is Omega-9 PUFA derivatives. In at least one embodiment, the Omega-9 PUFA derivatives are selected from cyclopropanated eicosenoic acid, cyclopropanated mead acid, cyclopropanated erucic acid, and cyclopropanated nervonic acid.

Yet another class of PKC-activating fatty acids is monounsaturated fatty acid (“MUFA”) derivatives. In at least one embodiment, the MUFA derivatives are selected from cyclopropanated oleic acid (shown below),

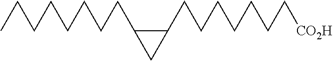 [SMILES: CCCCCCCCC1CC1CCCCCCCC(=O)O]

and cyclopropanated elaidic acid (shown below).

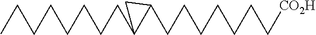 [SMILES: CCCCCCCCC1CC1CCCCCCCC(=O)O]

PKC-activating MUFA derivatives include epoxylated compounds such as trans-9,10-epoxystearic acid (shown below).

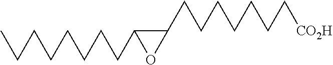 [SMILES: CCCCCCCCC1OC1CCCCCCCC(=O)O]

Another class of PKC-activating fatty acids is Omega-5 and Omega-7 PUFA derivatives. In at least one embodiment, the Omega-5 and Omega-7 PUFA derivatives are selected from cyclopropanated rumenic acid, cyclopropanated alpha-elostearic acid, cyclopropanated catalpic acid, and cyclopropanated punicic acid.

Another class of PKC activators is fatty acid alcohols and derivatives thereof, such as cyclopropanated PUFA and MUFA fatty alcohols. It is thought that these alcohols activate PKC by binding to the PS site. These alcohols can be derived from different classes of fatty acids.

In at least one embodiment, the PKC-activating fatty alcohols are derived from Omega-3 PUFAs, Omega-6 PUFAs, Omega-9 PUFAs, and MUFAs, especially the fatty acids noted above. In at least one embodiment, the fatty alcohol is selected from cyclopropanated linolenyl alcohol (CP3 form shown below),

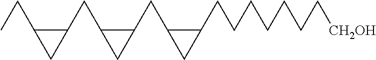 [SMILES: CCC1CC1CC1CC1CC1CC1CCCCCCCCO]

cyclopropanated linoleyl alcohol (CP2 form shown below),

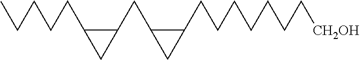 [SMILES: CCCCCC1CC1CC1CC1CCCCCCCCO]

cyclopropanated elaidic alcohol (shown below),

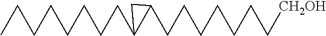 [SMILES: CCCCCCCCC1CC1CCCCCCCCO]

cyclopropanated DCPLA alcohol, and cyclopropanated oleyl alcohol.

Another class of PKC activators includes fatty acid esters and derivatives thereof, such as cyclopropanated PUFA and MUFA fatty esters. In at least one embodiment, the cyclopropanated fatty esters are derived from Omega-3 PUFAs, Omega-6 PUFAs, Omega-9 PUFAs, MUFAs, Omega-5 PUFAs, and Omega-7 PUFAs. These compounds are thought to activate PKC through binding on the PS site. One advantage of such esters is that they are generally considered to be more stable that their free acid counterparts.

In one embodiment, the PKC-activating fatty acid esters derived from Omega-3 PUFAs are selected from cyclopropanated eicosapentaenoic acid methyl ester (CP5 form shown below)

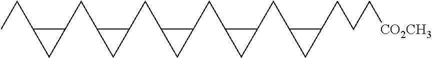 [SMILES: CCC1CC1CC1CC1CC1CC1CC1CC1CC1CC1CCCC(=O)OC]

and cyclopropanated linolenic acid methyl ester (CP3 form shown below).

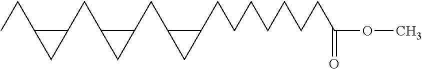 [SMILES: CCC1CC1CC1CC1CC1CC1CCCCCCCC(=O)OC]

In another embodiment, the Omega-3 PUFA esters are selected from esters of DHA-CP6 and aliphatic and aromatic alcohols. In at least one embodiment, the ester is cyclopropanated docosahexaenoic acid methyl ester (CP6 form shown below).

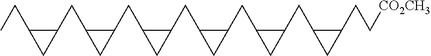 [SMILES: CCC1CC1CC1CC1CC1CC1CC1CC1CC1CC1CC1CC1CCC(=O)OC]

In one embodiment, PKC-activating fatty esters derived from Omega-6 PUFAs are selected from cyclopropanated arachidonic acid methyl ester (CP4 form shown below),

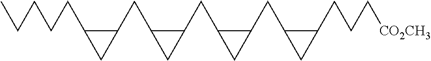 [SMILES: CCCCCC1CC1CC1CC1CC1CC1CC1CC1CCCC(=O)OC]

cyclopropanated vernolic acid methyl ester (CP1 form shown below), and

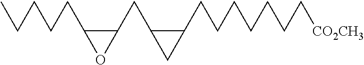 [SMILES: CCCCCC1OC1CC1CC1CCCCCCCC(=O)OC]

vernolic acid methyl ester (shown below).

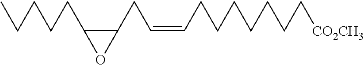 [SMILES: CCCCCC1OC1C/C=C\CCCCCCCC(=O)OC]

In particular embodiments, the PKC activating compound is an ester derivative of DCPLA (CP6-linoleic acid). In one embodiment, the ester of DCPLA is an alkyl ester. The alkyl group of the DCPLA alkyl esters may be linear, branched, and/or cyclic. The alkyl groups may be saturated or unsaturated. When the alkyl group is an unsaturated cyclic alkyl group, the cyclic alkyl group may be aromatic. The alkyl group may be selected from, for example, methyl, ethyl, propyl (e.g., isopropyl), and butyl (e.g., tert-butyl) esters. DCPLA in the methyl ester form (“DCPLA-ME”) is shown below.

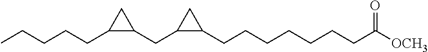 [SMILES: CCCCCC1CC1CC1CC1CCCCCCCC(=O)OC]

In another embodiment, the esters of DCPLA are derived from a benzyl alcohol (unsubstituted benzyl alcohol ester shown below). In yet another embodiment, the esters of DCPLA are derived from aromatic alcohols such as phenols used as antioxidants and natural phenols with pro-learning ability. Some specific examples include estradiol, butylated hydroxytoluene, resveratrol, polyhydroxylated aromatic compounds, and curcumin.

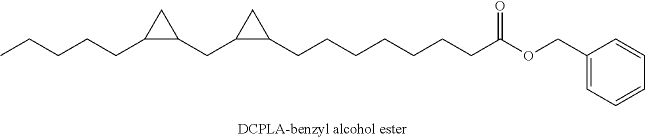 [SMILES: CCCCCC1CC1CC1CC1CCCCCCCC(=O)OCc1ccccc1]

Another class of PKC activators includes fatty esters derived from cyclopropanated MUFAs. In at least one embodiment, the cyclopropanated MUFA ester is selected from cyclopropanated elaidic acid methyl ester (shown below),

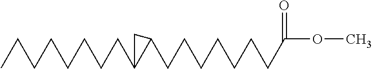 [SMILES: CCCCCCCCC1CC1CCCCCCCC(=O)OC]

and cyclopropanated oleic acid methyl ester (shown below).

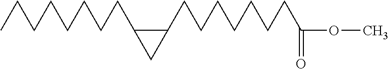 [SMILES: CCCCCCCCC1CC1CCCCCCCC(=O)OC]

Another class of PKC activators includes sulfates and phosphates derived from PUFAs, MUFAs, and their derivatives. In at least one embodiment, the sulfate is selected from DCPLA sulfate and DHA sulfate (CP6 form shown below).

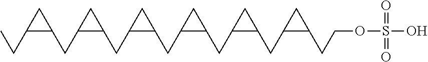 [SMILES: CCC1CC1CC1CC1CC1CC1CC1CC1CC1CC1CC1CC1CCOS(=O)(=O)O]

In one embodiment, the phosphate is selected from DCPLA phosphate and DHA phosphate (CP6 form shown below).

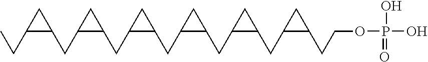 [SMILES: CCC1CC1CC1CC1CC1CC1CC1CC1CC1CC1CC1CC1CCOP(=O)(O)O]

In some embodiments, the PKC activator is selected from macrocyclic lactones, bryologs, diacylglycerols, isoprenoids, octylindolactam, gnidimacrin, ingenol, iripallidal, napthalenesulfonamides, diacylglycerol inhibitors, growth factors, polyunsaturated fatty acids, monounsaturated fatty acids, cyclopropanated polyunsaturated fatty acids, cyclopropanated monounsaturated fatty acids, fatty acids alcohols and derivatives, or fatty acid esters.

The PKC activators according to the present disclosure may be administered to a patient/subject in need thereof by conventional methods, such as oral, parenteral, transmucosal, intranasal, inhalation, or transdermal administration. Parenteral administration includes intravenous, intra-arteriolar, intramuscular, intradermal, subcutaneous, intraperitoneal, intraventricular, intrathecal, ICV, intracisternal injections or infusions and intracranial administration. A suitable route of administration may be chosen to permit crossing the blood-brain barrier. See e.g., J. Lipid Res. (2001) vol. 42, pp. 678-685, incorporated by reference herein.

The PKC activators can be compounded into a pharmaceutical composition suitable for administration to a subject using general principles of pharmaceutical compounding. In one aspect, the pharmaceutically acceptable composition comprises a PKC activator and a pharmaceutically acceptable carrier.

The formulations of the compositions described herein may be prepared by any suitable method known in the art. In general, such preparatory methods include bringing at least one of the active ingredients into association with a carrier. If necessary or desirable, the resultant product can be shaped or packaged into a desired single-or multi-dose unit.

As discussed herein, carriers include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other additional ingredients that may be included in the compositions of the disclosure are generally known in the art and may be described, for example, in Remington's Pharmaceutical Sciences, Genaro, ed., Mack Publishing Co., Easton, Pa., 1985, and Remington's Pharmaceutical Sciences, 20^{th} Ed., Mack Publishing Co. 2000, both incorporated by reference herein.

In at least one embodiment, the carrier is an aqueous or hydrophilic carrier. In a further embodiment, the carrier can be water, saline, or dimethylsulfoxide. In another embodiment, the carrier is a hydrophobic carrier. Hydrophobic carriers include inclusion complexes, dispersions (such as micelles, microemulsions, and emulsions), and liposomes. Exemplary hydrophobic carriers include inclusion complexes, micelles, and liposomes. See, e.g., Remington's: The Science and Practice of Pharmacy 20th ed., ed. Gennaro, Lippincott: Philadelphia, PA 2003, incorporated by reference herein. In addition, other compounds may be included either in the hydrophobic carrier or the solution, e.g., to stabilize the formulation.

In some embodiments, the compositions described herein may be formulated into oral dosage forms. For oral administration, the composition may be in the form of a tablet or capsule prepared by conventional means with, for example, carriers such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets may be coated by methods generally known in the art.

In another embodiment, the compositions herein are formulated into a liquid preparation. Such preparations may be in the form of, for example, solutions, syrups or suspensions, or they may be presented as a dry product for constitution with water or other suitable vehicle before use. Such liquid preparations may be prepared by conventional means using pharmaceutically acceptable carriers, such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl p-hydroxybenzoates, or sorbic acid). The preparations may also comprise buffer salts, flavoring, coloring, and sweetening agents as appropriate. In some embodiments, the liquid preparation is specifically designed for oral administration.

In another embodiment of the present disclosure, the compositions herein may be formulated for parenteral administration such as bolus injection or continuous infusion. Formulations for injection may be presented in unit dosage form, e.g., in ampoules, or in multi-dose containers, with an added preservative. The composition may be in the form of a suspension, solution, dispersion, or emulsion in oily or aqueous vehicles, and may contain a formulary agent, such as a suspending, stabilizing, and/or dispersing agent.

In another embodiment, the compositions herein may be formulated as depot preparations. Such formulations may be administered by implantation (for example, subcutaneously or intramuscularly) or by intramuscular injection. For example, the compositions may be formulated with a suitable polymeric or hydrophobic material (for example, as an emulsion in an acceptable oil) or ion exchange resin, or as a sparingly soluble derivative, for example, as a sparingly soluble salt.

In another embodiment, at least one PKC activator or combination thereof is delivered in a vesicle, such as a micelle, liposome, or an artificial low-density lipoprotein (LDL) particle. See, e.g., U.S. Pat. No. 7,682,627, the contents of which are herein incorporated by reference.

In some embodiments, at least one PKC activator or combination of PKC activators may be present in the pharmaceutical composition in an amount ranging from about 0.01% to about 100%, from about 0.1% to about 90%, from about 0.1% to about 60%, from about 0.1% to about 30% by weight, or from about 1% to about 10% by weight of the final formulation. In another embodiment, at least one PKC activator or combination of PKC activators may be present in the composition in an amount ranging from about 0.01% to about 100%, from about 0.1% to about 95%, from about 1% to about 90%, from about 5% to about 85%, from about 10% to about 80%, and from about 25% to about 75%.

The present disclosure further relates to kits that may be utilized for administering to a subject a PKC activator according to the present disclosure. The kits may comprise devices for storage and/or administration. For example, the kits may comprise syringe(s), needle(s), needle-less injection device(s), sterile pad(s), swab(s), vial(s), ampoule(s), cartridge(s), bottle(s), and the like. The storage and/or administration devices may be graduated to allow, for example, measuring volumes. In at least one embodiment, the kit comprises at least one PKC activator in a container separate from other components in the system.

The kits may also comprise one or more anesthetics, such as local anesthetics. In at least one embodiment, the anesthetics are in a ready-to-use formulation, for example an injectable formulation (optionally in one or more pre-loaded syringes), or a formulation that may be applied topically. Topical formulations of anesthetics may be in the form of an anesthetic applied to a pad, swab, towelette, disposable napkin, cloth, patch, bandage, gauze, cotton ball, Q-tip™, ointment, cream, gel, paste, liquid, or any other topically applied formulation. Anesthetics for use with the present disclosure may include, but are not limited to lidocaine, marcaine, cocaine, and xylocaine.

The kits may also contain instructions relating to the use of at least one PKC activator or a combination thereof. In another embodiment, the kit may contain instructions relating to procedures for mixing, diluting, or preparing formulations of at least one PKC activator or a combination thereof. The instructions may also contain directions for properly diluting a formulation of at least one PKC activator or a combination thereof in order to obtain a desired pH or range of pHs and/or a desired specific activity and/or protein concentration after mixing but prior to administration. The instructions may also contain dosing information. The instructions may also contain material directed to methods for selecting subjects for treatment with at least one PKC activator or a combination thereof.

The PKC activator can be formulated, alone in suitable dosage unit formulations containing conventional non-toxic pharmaceutically acceptable carriers, adjuvants and vehicles appropriate for each route of administration. Pharmaceutical compositions may further comprise other therapeutically active compounds which are approved for the treatment of neurodegenerative diseases or to reduce the risk of developing a neurodegenerative disorder.

All of the references, patents and printed publications mentioned in the instant disclosure are hereby incorporated by reference in their entirety into this application.

The following examples are provided by way of illustration to further describe certain preferred embodiments of the invention, and are not intended to be limiting of the present disclosure.

### EXAMPLES

Mouse studies may be performed using bryostatin-1 or other PKC activating compound, as described above. An hsOD1 transgenic mouse model (SOD1), in particular, may be used. SOD1, which overexpresses the human SOD1G93A mutation, is one of the most definitive animal models of ALS (M. J. Fogarty et al., Frontiers in Neuroscience, 11(609), November 2017). SOD1 mutations are found in 10-20% of familial ALS and in 1-2% of sporadic ALS cases. A modified Golgi-Cox staining method may be used to determine the progressive changes in dendritic structure of hippocampal CA1 pyramidal neurons, striatal medium spiny neurons, and resistant (trochlear, IV) or susceptible (hypoglossal, XII; lumbar) motor neurons from brainstem and spinal cord of mice over-expressing the human SOD1 mutation. The changes may be compared to wild type (WT) mice, e.g., at four post-natal (P) ages of 8-15, 28-35, 65-75, and 120 days. According to protocols discussed below, there may be observed a mitigation or halting of the degeneration of motor neurons of the spinal cord, medulla, and/or cortex, or even regeneration of any of these types of motor neurons.

Groups of 2-3 mice may be formed and housed in an approved research animal facility. Water may be given ad libitum. A first study involves three groups of mice with animals in each group dosed weekly for 1, 2, 3, or 6 consecutive weeks. Each group has its own control group containing the same number of mice. For example, mice in the first, second and third groups may receive an intravenous (i.v.) injection of 10 μg/m², 15 μg/m², and 25 μg/m² dose of bryostatin or other PKC activating compound respectively. For each dose, mice in that group may receive a single injection of a bryostatin or other PKC activating compound weekly for a predetermined number of consecutive weeks. Following dosing, mice are sacrificed and the blood and brain of each animal is collected for further analysis.

In some cases, mice are dosed weekly with bryostatin or other PKC activating compound at about 25 μg/m² for three consecutive weeks, followed by cessation of drug administration for three consecutive weeks, and then a second round of dosing at about 25 μg/m² for an additional three consecutive weeks (that is, a “3 on/3 off/3 on” dosing regimen). In other embodiments, mice are dosed at about 25 μg/m² at a “1 on/1 off” regimen for a total of nine weeks (e.g., one dose of bryostatin or other PKC activating compound on weeks 1, 3, 5, 7, and 9, with no dosing in weeks 2, 4, 6, and 8). In other embodiments, mice are dosed at about 25 μg/m² for another regimen starting with “2 on/1 off” immediately followed by alternating “1 on/1 off” until reaching the ninth total week (i.e., one dose of bryostatin or other PKC activating compound on weeks 1, 2, 4, 6, 8, with no dosing in weeks 3, 5, 7, and 9). Increasing the duration of the rest intervals (i.e., “off” intervals) to three weeks may significantly reduce PKC downregulation. That is, the “3 on/3 off” dosing regimen may increase brain PKC-ε levels in mice over the other regimens, thus resulting in particularly beneficial results.

Brain BDNF in mice may reach its highest level after three consecutive weekly doses of bryostatin or other PKC activating compound at about 25 μg/m² and remain elevated after three additional consecutive weeks of no dosing, followed by three more consecutive weekly doses at about 25 μg/m². Since BDNF is a peptide that induces synaptogenesis (i.e., the formation of new synapses), a “3 on/3 off” regimen may maximize synaptogenesis and minimize PKC downregulation.

Further evaluation may be performed on bryostatin or other PKC activating compound crossing the blood-brain-barrier (BBB) and the steady state levels of bryostatin or other PKC activating compound in the brain and plasma of mice. In some embodiments, bryostatin or other PKC activating compound administered intravenously crosses the BBB. In that case, the concentration of bryostatin or other PKC activating compound in mice brain may be less than its concentration in plasma. However, the concentration in brain may be no less than two-fold lower than the plasma concentrations for comparable doses under steady-state conditions.

A weekly dosing regimen of a single injection of bryostatin or other PKC activating compound at a dose of about 25 μg/m² for three consecutive weeks may be less effective at increasing bryostatin concentration or other PKC activating compound in mice brain than a “1 on/1 off” or a “2 on/1 off” administration of the 25 μg/m² dose. In contrast, plasma concentrations of bryostatin or other PKC activating compound may be greater when the drug is administered as a single injection for three consecutive weeks. Blood plasma concentrations of bryostatin or other PKC activating compound may be less in mice receiving a 25 μg/m² dose as a “1 on/1 off” or a “2 on/1 off” administration. Without being bound to a specific theory, it may be hypothesized that the intermittent dosing regimen facilitates the transport of bryostatin or other PKC activating compound across the BBB.

While there have been shown and described what are at present considered the preferred embodiments of the invention, those skilled in the art may make various changes and modifications which remain within the scope of the invention defined by the appended claims.

1. A method for treating amyotrophic lateral sclerosis (ALS) in a subject in need thereof, the method comprising administering to said subject a pharmaceutically effective amount of a PKC activator selected from the group consisting of polyunsaturated fatty acids, esters thereof, cyclopropanated derivatives thereof, epoxidized derivatives thereof, and pharmaceutically acceptable salts thereof.

2. The method of claim 1, wherein said ALS is selected from the group consisting of classical ALS, primary lateral sclerosis (PLS), progressive muscular atrophy (PMA), and progressive bulbar palsy (PBP).

3.-8. (canceled)

9. The method of claim 1, wherein the PKC activating compound is a cyclopropanated polyunsaturated fatty acid ester having the following structure: 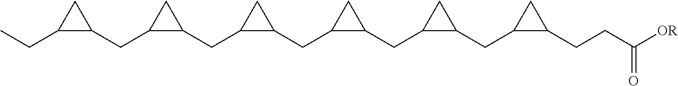
wherein R is an alkyl group.

10.-11. (canceled)

12. The method of claim 1, wherein the PKC activating compound is administered intravenously.

13. The method of clam 1, wherein the PKC activating compound is administered as an oral dosage form.

14. The method of claim 1, wherein the PKC activating compound is administered in an amount of 10-50 μg/m² weekly for at least 1 week.

15. The method of claim 1, wherein the PKC activating compound is administered in an amount of 10-50 μg/m² weekly for at least 3 weeks.

16. The method of claim 1, wherein the PKC activating compound is administered in an amount of 20-50 μg/m² weekly for at least 1 week.

17. The method of claim 1, wherein the PKC activating compound is administered in an amount of 20-50 μg/m² weekly for at least 3 weeks.

18. The method of claim 1, wherein the PKC activating compound is administered in an amount of 20-40 μg/m² weekly for at least 1 week.

19. The method of claim 1, wherein the PKC activating compound is administered in an amount of 20-40 μg/m² weekly for at least 3 weeks.

20. The method of claim 1, wherein the PKC activating compound is administered in an amount of 30-50 μg/m² weekly for at least 1 week.

21. The method of claim 1, wherein the PKC activating compound is administered in an amount of 30-50 μg/m² weekly for at least 3 weeks.

22. The method of claim 1, wherein the PKC activating compound is administered in an amount of 25-40 μg/m² weekly for at least 1 week.

23. The method of claim 1, wherein the PKC activating compound is administered in an amount of 25-40 μg/m² weekly for at least 3 weeks.

24. The method of claim 1, wherein the PKC activating compound is administered at an initial loading dose of about 15 micrograms per week for two consecutive weeks followed by about 12 micrograms on alternate weeks for a least four weeks.

25. The method of claim 1, wherein the PKC activating compound is administered at an initial loading dose of about 24 micrograms per week for two consecutive weeks followed by about 20 micrograms on alternate weeks for a least four weeks.

26. The method of claim 1, wherein the PKC activating compound is administered at an initial loading dose of about 48 micrograms per week for two consecutive weeks followed by about 40 micrograms on alternate weeks for a least four weeks.
